(19) United States
(12) Patent Application Publication
(10) Pub. No.: US 2012/0308433 A1
(43) Pub. Date: Dec. 6, 2012

(54) TREATMENT OF CHEMICAL/BIOLOGICAL WARFARE AGENTS

(75) Inventor: Nicholas Mark Turner Adams, Clanville (GB)

(73) Assignee: BIOQUELL UK LIMITED, Andover, Hampshire (UK)

(21) Appl. No.: 13/577,626

(22) PCT Filed: Feb. 24, 2011
(86) PCT No.: PCT/GB2011/000254

§ 371 Date: Aug. 7, 2012

(30) Foreign Application Priority Data
Feb. 24, 2010 (GB) 1003126.8
Sep. 22, 2010 (GB) 1015950.7
Oct. 27, 2010 (GB) 1018173.3

(51) Int. Cl. A61L 2/20

(52) U.S. Cl. 422/28

(57) ABSTRACT

A method of rendering harmless chemical and/or biological warfare agents on a surface, comprising: (i) exposing the agents to an atmosphere comprising peroxide vapour, and causing the peroxide vapour to condense on the surface having the chemical and/or biological agents thereon; (ii) after step (i) exposing the agents to an atmosphere comprising further peroxide vapour, and causing the further peroxide vapour to condense on the surface having the chemical and/or biological agents thereon; and (iv) optionally repeating step (ii).

The present invention relates to a method of decontaminating surfaces which have been exposed to biological and/or chemical warfare agents.

WO 2008/145987 describes a method of rendering harmless chemical/biological warfare agents. This comprises exposing the agents to an atmosphere which includes a peroxide/water vapour, and causing the peroxide vapour to condense on surfaces exposed to the chemical/biological agents; wherein ammonia gas is included in the atmosphere. The ammonia gas is soluble in the condensate to form ammonia hydroxide to react, in conjunction with the peroxide, against the biological/chemical agents.

U.S. Pat. No. 7,102,052 B2 and the International Patent Application WO 2005/035067 A2 describe in detail a method of decontaminating surfaces that have been contaminated with both biological and chemical agents. The chemistry of the chemical decontamination using hydrogen peroxide vapour mixed with ammonia gas or other nitrogen containing compounds is fully explained. The advantages of the techniques described in the US Patent and the International Patent Application are said to be that the gases may be applied either from an external source to an enclosure or generated within the enclosure, that there is no residue or liquid to be removed at the end of the decontamination procedure and because it is a gaseous process, of which the only by-products are oxygen and water vapour, no damage will occur to sensitive equipment. The disadvantage of the described process is that according to the data given in the International application decontamination of chemical agent VX takes up to 24 hours. Such a long time period is undesirable especially if a significant number of items of equipment need decontamination; any technique that may shorten this period would offer advantages especially when faced with repeated or widespread chemical and biological attacks.

It is one object of the present invention to provide an improved method of rendering harmless chemical/biological warfare agents on a surface. Preferably, the method provides a process which takes a shorter period of time to render harmless chemical/biological warfare agents on a surface and is more efficient than prior art methods. It is also an object to provide a commercially viable alternative to known techniques of rendering chemical/biological warfare agents harmless.

In the first aspect of the present invention there is provided a method of rendering harmless chemical and/or biological warfare agents on a surface, comprising:
(i) exposing the agents to an atmosphere comprising peroxide vapour, and causing the peroxide vapour to condense on the surface having the chemical and/or biological agents thereon;
(ii) after step (i) exposing the agents to an atmosphere comprising further peroxide vapour, and causing the further peroxide vapour to condense on the surface having the chemical and/or biological agents thereon
(iii) optionally repeating step (ii).

The present invention will now be further described. In the following passages different aspects of the invention are defined in more detail. Each aspect so defined may be combined with any other aspect or aspects unless clearly indicated to the contrary. In particular any feature indicated as being preferred or advantageous may be combined with any other feature or features indicated as being preferred or advantageous.

After step (i) the agents are exposed to an atmosphere comprising further peroxide vapour, and peroxide vapour is caused to condense on the surface having the chemical/biological agents thereon. As used herein the term “an atmosphere comprising further peroxide vapour” means an atmosphere comprising peroxide vapour which has been added in the new step and which was not present as a result of the previous step. In summary, the “further peroxide vapour” is fresh, new peroxide vapour which was not present in the previous step, or as a result of the previous step.

The present invention does not include a situation where the agents are continuously exposed to a flow of fresh peroxide vapour. In this method, step (i), step (ii) and optionally further step (ii)s are distinct (or discrete) steps, and there is a time period between the steps in which time the condensed peroxide vapour dwells on the surface to be decontaminated without further fresh peroxide being added. Typically, in each step, the atmosphere comprising peroxide vapour is introduced into an enclosure or system over a time period of from 2 to 30 minutes. Typically, the time period between each step (i.e. the time period during which no further peroxide is added to the enclosure or system) is from 5 to 60 minutes, or from 10 to 30 minutes. Typically, the time period between each of the discrete steps (i), (ii) and optionally repeated steps (ii) is independently at least 1 minute, at least 5 minutes, at least 10 minutes or at least 30 minutes.

As outlined in WO 2008/145987, it is well known that biological decontamination is faster once hydrogen peroxide and water vapours have reached saturation and a fine layer of condensation has formed. The reason is that the number of molecules available to attack the microorganisms is much greater in the liquid phase than in the vapour phase. Also, in the liquid phase the contact time of each molecule with the target microorganism is much longer because of the lower kinetic energy of the molecules in the liquid state. The same argument applies to chemical decontamination as biological decontamination is effectively a chemical reaction. Faster reactions will take place once the vapours have become saturated and condensation has formed.

The present inventors have surprisingly found that unlike in the prior art methods where typically a surface to be decontaminated is exposed only once to an excess of peroxide and then the peroxide is removed with base, the present inventors have found that repeating exposure of the surface-to-be-decontaminated to further, fresh peroxide vapour allows a new fresh layer of peroxide to be condensed onto the surface. Such a method has been found to lead to a more efficient and effective decontamination of the surface. As will be appreciated, any method which can more quickly and effectively decontaminate a surface is desirable over slower, less efficient methods. Repeating step (ii) allows a fresh layer of peroxide condensate to be added to the surface to be decontaminated and thus allows the peroxide concentration on the surface to be replenished.

Without wishing to be bound by theory it is thought that when the peroxide is condensed onto a surface, it is at least partially consumed in the reaction with the biological/chemical agent in addition to its concentration decreasing due to the natural breakdown of the molecule. It has been found that repeated exposure of the surface to peroxide is an effective method of replenishing the number of moles of hydrogen peroxide present in the enclosure or system. This provides a supply of peroxide molecules to react with the agent.

In order to further maximise the efficiency and effectiveness of the decontamination process, step (ii) is preferably repeated at least once, (i.e. performed twice), preferably repeated twice (i.e. performed three times), and more preferably repeated three or more times.

Repeating the steps significantly speeds up the decontamination process by keeping the concentrations of hydrogen peroxide high. This leads to a higher concentration of the oxidising species/radicals being present on the surface and therefore leads to a faster reaction with the chemical warfare agents.

In one embodiment there is provided a method of rendering harmless chemical and/or biological warfare agents on a surface, consisting of the following steps:
(i) exposing the agents to an atmosphere comprising peroxide vapour, and causing the peroxide vapour to condense on the surface having the chemical and/or biological agents thereon;
(ii) after step (i) exposing the agents to an atmosphere comprising further peroxide vapour, and causing the further peroxide vapour to condense on the surface having the chemical and/or biological agents thereon; and
(iii) optionally repeating step (ii).

In one embodiment of the present invention the method further comprises after step (i) adding gaseous base such that the gaseous base reacts with the condensed peroxide vapour on the surface. Such a method is of particular use for rendering harmless chemical warfare agents, for example, one or more of a G-type agent and a V-type agent.

In another embodiment of the present invention, preferably the surface is not basic, and base is not present in significant amounts (less than 35 ppm), and is preferably not present, when steps (i) and (ii) are carried out. Preferably no ammonia or base is present in the system. In one embodiment of the present invention, the method does not comprise adding gaseous base and/or base. In one embodiment no or substantially no alkaline compounds are added and/or are present (for example when steps (i) and (ii) are carried out). Preferably no ammonia is present or added.

Preferably, no gaseous base having the formula NR¹R²R³, wherein R¹, R² and R³ are as defined below is added in the method described herein or is present (for example when steps (i) and (ii) are carried out). One advantage of this embodiment is that the method does not require ammonia scrubbing. This embodiment is of particular use when the chemical and/or biological warfare agents to be rendered harmless by the method of the present invention are biological warfare agents and/or include one or more of a H-type agent, pathogens, biotoxins, spores and prions.

In a preferred embodiment the present invention provides a method of rendering harmless chemical and/or biological warfare agents on a surface, comprising:
(I) exposing the agents to an atmosphere comprising peroxide vapour, and causing the peroxide vapour to condense on the surface having the chemical/biological agents thereon;
(II) after step (I) adding a gaseous base such that the gaseous base reacts with the condensed peroxide vapour on the surface;
(III) removing the gaseous base;
(IV) repeating steps (I) and (II) at least once.

Thus, in one embodiment, step (i) corresponds (or is equal) to step (I), and step (ii) corresponds (or is equal) to the repeating step (I) in step (IV).

In one embodiment there is provided a method of rendering harmless chemical and/or biological warfare agents on a surface, consisting of the following steps:
(I) exposing the agents to an atmosphere comprising peroxide vapour, and causing the peroxide vapour to condense on the surface having the chemical/biological agents thereon;
(II) after step (I) adding a gaseous base such that the gaseous base reacts with the condensed peroxide vapour on the surface;
(III) removing the gaseous base;
(IV) repeating steps (I) and (II) at least once.

The present inventors have surprisingly found that unlike in the prior art methods where typically peroxide and/or ammonia is added simultaneously in a one-step reaction to decontaminate surfaces, the inventors have found the decontamination process is significantly more efficient if the steps are carried out in a stepwise manner and repeated. This is thought to be because the base, which is preferably ammonia, catalytically decomposes the peroxide. Thus, adding the two reagents together causes a dramatic reduction in peroxide concentration. The less peroxide present in the system, the less decontamination, as in this embodiment it is thought to be the peroxide in the basic environment which reacts with the chemical/biological warfare agents.

Without wishing to be bound by any particular theory, it is thought that if peroxide vapour is caused to condense on a surface in the presence of gaseous base, the activity of the peroxide is reduced. This is a result of the ammonia breaking down the peroxide before surface decontamination has occurred. Thus, if the gaseous base (preferably ammonia) and the peroxide are added simultaneously, the decontamination process is significantly less efficient that if peroxide is condensed onto the surface to be decontaminated, and gaseous base (preferably ammonia gas) is subsequently added.

Some prior art methods suggest exposing the surface to be decontaminated first to peroxide and then to ammonia. However, there is no disclosure of subsequently repeating these steps.

The present inventors have found that in order to further maximise the efficiency and effectiveness of the decontamination process, steps (I) and (II), and preferably also step (III), are repeated at least once, (i.e. performed twice), preferably they are repeated twice (i.e. performed three times), and more preferably still they are repeated three or more times.

A further unexpected advantage of repeating steps (I) and (II) and preferably step (III) in an iterative, or pulsed manner, is that the total amount of peroxide and/or gaseous base required for decontamination is less than the total amounts used in the prior art methods in order to achieve the same level of chemical/biological warfare agent decontamination. This is surprising in view of the need to repeat the process steps. However, it is a result of the method described herein solving the problem of deactivation of the peroxide vapour by the gaseous base prior to rendering the chemical/biological warfare agent on the surface harmless. Thus, in the present invention the peroxide vapour is more efficiently used. Repeating the steps allows the peroxide concentration to be replenished.

It has surprisingly been found that by using the method of the present invention decontamination of the surface may be achieved in less than 5 hours, preferably, less than 2 hours, less than 1 hour, and most preferably less than 45 minutes.

Thus, the present invention aims to provide an improved method of decontaminating surfaces comprising chemical/biological warfare agents in which in the first step the peroxide vapour is caused to condense on the surface exposed to the chemical/biological warfare agents.

This may be achieved by providing conditions in which the concentration of the peroxide vapour is increased until the dew point of the vapour is exceeded and condensation of the vapour on surfaces takes place. Such methods are known in the art, see for example WO 2008/145987.

Any suitable peroxide may be used in the present method. For example, the peroxide may be selected from one or more peroxy compounds such as hydrogen peroxide, peracetic acid and mixtures thereof. Preferably, the peroxide used in the present invention is hydrogen peroxide.

Preferably the peroxide vapour comprises water. Typically, the peroxide vapour is present as a combination of water vapour and peroxide vapour.

The peroxide chosen is preferably capable of forming free radicals required to oxidise the chemical/biological agent.

Any appropriate concentration of peroxide may be used in the present invention. Typically hydrogen peroxide will be used in a concentration of 30% w/w in water. However, hydrogen peroxide may be used in a concentration of for example from 10% w/w to 75% w/w in water, or from 20% w/w to 45% w/w in water.

The chemical/biological warfare agents which may be rendered harmless by the method of the present invention include one or more of a G-type agent, a V-type, a H-type agent, pathogens, biotoxins, spores and prions.

Preferably the surface having the chemical and/or biological agents thereon is provided in an enclosure and/or is at least part of an enclosure. The method of the present invention may comprise the step of placing the surface having the chemical and/or biological agents thereon in an enclosure before carrying out the decontamination process. For example, this may occur when the surface to be treated is a garment or piece of clothing, breathing apparatus, weaponry, medical instruments or any other surface, or apparatus comprising a surface suitable for placing in an enclosure. The enclosure is preferably a container or chamber, preferably a treatment chamber. In another example, the surface to be decontaminated may comprise at least part of the enclosure. For example the surface to be treated may be, for example, the interior of a warehouse, tent, room, aircraft, tank, other vehicle, or the like, whose surfaces (preferably interior surfaces) or items are to be treated. Preferably the enclosure is capable of being made air-tight. It will be understood that suitable methods know in the art may be utilised to make the enclosure air-tight.

As used herein the term “enclosure comprising the surface” includes wherein the surface having the chemical and/or biological agents thereon is provided in an enclosure and/or is at least part of an enclosure. For example the surface to be treated may be a wall of a room.

Preferably, in step (i) the enclosure comprising the surface is exposed to an atmosphere comprising from 15 to 180 g of peroxide per m³ of volume of the enclosure.

Preferably in step (ii) enclosure comprising the surface is exposed to an atmosphere comprising from 5 to 75 g of peroxide per m³ of volume of the enclosure.

Step (ii) may be repeated once, twice, three, four or more times.

In one embodiment of the present invention, each time step (ii) is repeated, the enclosure comprising the surface is exposed to a reduced amount of peroxide compared to the amount of peroxide to which the enclosure comprising the surface is exposed in the immediately preceding step (i) measured in g of peroxide per m³ of volume of the enclosure.

By carrying out the process in an iterative way, and each time decreasing the amount of peroxide used the decontamination of the surface may be optimised, whilst making efficient use of the peroxide.

In one embodiment each time step (ii) is repeated, the enclosure comprising the surface is exposed to the same amount of peroxide compared to the amount of peroxide to which the enclosure comprising the surface is exposed in the immediately preceding step (ii) measured in g of peroxide per m³ of volume of the enclosure.

Preferably, in steps (i) and (ii) the enclosure comprising the surface comprising the chemical/biological warfare agent is exposed to a pulse of peroxide with a volume dependent on the level of equipment loading in the enclosure (for example a chamber). A lightly loaded enclosure, for example a chamber, typically requires from 15 to 50 g of peroxide per cubic metre of enclosure to be decontaminated. Typically a medium loaded enclosure, for example a chamber, is exposed to from 50 to 75 g of peroxide per cubic metre, and a heavily loaded enclosure, for example a chamber, is typically exposed to between 75 to 150 g per cubic metre of enclosure to be decontaminated.

Preferably, the surface is exposed to an atmosphere comprising peroxide vapour for less than 1 hour, or less than 40 minutes. Typically the surface is exposed to an atmosphere comprising peroxide vapour for approximately 8 to 60 minutes, from 10 to 25, 30 to 35 minutes, more preferably about 10 minutes. It may be desirable to expose the surface to an atmosphere comprising peroxide vapour for short dwell times in order for the cycle time for the process to be fast.

As outlined above, in one embodiment of the present invention the method further comprises adding gaseous base. Preferably gaseous base is added such that the gaseous base reacts with the condensed peroxide vapour on the surface. Preferably, the gaseous base is added after step (i). Preferably the method further comprises removing at least some, and preferably all, of the gaseous base.

In particular, as outlined above, in a preferred embodiment the present invention provides a method of rendering harmless chemical and/or biological warfare agents on a surface, comprising:
(I) exposing the agents to an atmosphere comprising peroxide vapour, and causing the peroxide vapour to condense on the surface having the chemical/biological agents thereon;
(II) after step (I) adding a gaseous base such that the gaseous base reacts with the condensed peroxide vapour on the surface;
(III) removing the gaseous base;
(IV) repeating steps (I) and (II) at least once.

Preferably, this is embodiment, the method is designed such that deprotonation of the peroxide by gaseous base (preferably ammonia) occurs on the surface to be decontaminated whilst the peroxide is condensed on said surface.

In this embodiment of the present invention the peroxide is chosen to be capable of forming a peroxyl radical (·⁻OOR) where R can be hydrogen or any other alkyl group that may be substituted or unsubstituted upon reaction with the gaseous base.

The molar ratio of peroxide:gaseous base may be from 1:0.10 to 1:0.75, or approximately 1:0.15. However, more preferably, the molar ratio of peroxide:gaseous base is from 1:0.01 to 1:1, more preferably still, it is approximately 1:0.03. The base, typically ammonia, is thought to catalytically break down peroxide. Thus, less than a stoichiometric amount of base to peroxide is required in the present invention. However, it will be understood, that higher stoichiometric amounts of base may also be used if desired.

Preferably, after step (I) gaseous base is added such that base reacts with the condensed peroxide vapour on the surface.

It is advantageous that the base is added in the form of a gas because this allows for good distribution over the surface to be decontaminated.

Preferably the gaseous base has the formula NR¹R²R³, wherein R¹, R², R³ are independently selected from the group consisting of a C₁ to C₄ alkyl and hydrogen. The alkyl group may be substituted or un-substituted. Suitable substituents are those which do not significantly adversely affect the catalytic activity of the nitrogen containing compound. Preferably, NR¹R²R³ is selected from one or more of N(CH₃)₃, N(CH₂CH₂CH₃)₃, N(CH₂CH₃)₃, NH(CH₂CH₂CH₃)₂, NH(CH₂CH₂CH₃) (CH₂CH₃), NH(CH₂CH₃) (CH₃), NH(CH₂CH₃)₂, NH(CH₃)₂ and NH₃. Most preferably NR¹R²R³ is NH₃ (ammonia).

Most preferably, the gaseous base is ammonia and the peroxide is hydrogen peroxide, preferably having a concentration of 30% w/w in water.

Preferably at least some of the gaseous base dissolves into the peroxide vapour condensed on the surface. Preferably, after addition of the base, the condensed layer on the surface has a pH in the range of from about 9 to 13 or 14, preferably from about 9.5 to about 11.5 or higher, more preferably from about 10 to about 11 or higher and most preferably above 11. The present inventors have found that if the pH of the condensed vapour is high (for example in the ranges outlined above), then the decontamination process is faster than if the vapour is more acidic (less basic). The pH of the surfaces may be tested using pH indicator paper or any other suitable method. Thus, preferably the method described herein comprises adding the gaseous base such that the gaseous base reacts with the condensed peroxide vapour on the surface such that the pH of the condensed vapour is in the range of from about 9.5 to 13 or 14, from about 9.5 to about 11.5 or higher, more preferably from about 10 to about 11 or higher and most preferably the pH is above 11.

Preferably when step (I) is repeated the agents are exposed to an atmosphere comprising further peroxide vapour, and peroxide vapour is caused to condense on the surface having the chemical/biological agents thereon. Preferably when step (II) is repeated further gaseous base is added such that the gaseous base reacts with the condensed peroxide vapour on the surface. Preferably as used herein the term “an atmosphere comprising further peroxide vapour” means an atmosphere comprising peroxide vapour which has been added in the new step and which was not present as a result of the previous step. Preferably the term “further gaseous base” means gaseous base which has been added in the new step and which was not present as a result of the previous step. In summary, preferably the “further peroxide vapour” is fresh, new peroxide vapour which was not present in the previous step, or as a result of the previous step. Preferably the “further gaseous base” is fresh, new gaseous base which was not present in the previous step, or as a result of the previous step.

Steps (I), (II) and/or (III) are preferably discrete, separate steps. Preferably, the time period between each of the discrete/separate steps (I), (II) and/or (III) is independently at least 1 minute, at least 5 minutes, at least 10 minutes or at least 30 minutes.

In one embodiment of the present invention, after carrying out steps (I) and (II) and before repeating these steps, the gaseous base is removed from the enclosure or system. Preferably after removal of the gaseous base, less than 10% by volume, less than 5% by volume, less than 2% by volume, more preferably less than 1% by volume, more preferably still no gaseous base is left in the enclosure or system (chamber, if the reaction is carried out in a chamber, or in the immediate vicinity or the surface if, for example, the surface provides its own chamber, for example the surface is a room to be decontaminated) based on the total amount of gaseous base added in step (II). Most preferably, all the gaseous base is removed. The base gas may be at least partially removed by scrubbing. It may be preferable for as much of the gaseous base as possible to be removed from the system before the fresh peroxide is added. This minimizes any decomposition of peroxide by the base before the peroxide has condensed onto the surface to be decontaminated.

The amount of gaseous base present in the enclosure, system or chamber may be measured using any known method, for example using an electrochemical sensor, gas chromatography or infrared absorbance. A known volume of base is added to the enclosure, system or chamber. The volume of base in the enclosure, system or chamber after step (III) may be measured using standard techniques, thus the volume of gaseous base remaining after removal from the enclosure, system or chamber may be easily calculated.

Methods of scrubbing to remove basic gases, and in particular ammonia are known in the art and include, for example aerating the chamber through a chemical filter (typically activated carbon doped with CuCl₂). This scrubs the gas (i.e. ammonia) and breaks down any residual peroxide. After this aeration period more peroxide may be added.

In this embodiment, after the first time the gaseous base is removed in step (III), steps (I) and (II) at least are repeated. Repeating the steps significantly speeds up the decontamination process by keeping the concentrations of hydrogen peroxide high. This leads to a higher concentration of the ·OOH⁻ reactive intermediate being present on the surface and therefore leads to a faster reaction with the chemical warfare agents.

Preferably after repeating steps (I) and (II), the gaseous base is removed from the enclosure or system once more. Preferably after removal of the gaseous base, less than 10% by volume, less than 5% by volume, less than 2% by volume, more preferably less than 1% by volume, more preferably still no gaseous base is left in the enclosure or system (chamber, if the reaction is carried out in a chamber, or in the immediate vicinity or the surface if for example the surface provides its own chamber, for example the surface is a room to be decontaminated) based on the total amount of gaseous base added in step (II). Most preferably, all the gaseous base is removed.

In another embodiment of the invention, steps (I) and (II) are repeated, at least twice, preferably at least three times, before step (III) is carried out. The inventors have found that it is advantageous to first expose the chemical and/or biological agents on a surface to an atmosphere comprising peroxide vapour, causing the peroxide vapour to condense onto the surface. This first step is preferably carried out in the absence of a base. As outlined above, this first exposure of the surface having chemical and/or biological agents thereon preferably renders at least some of the chemical and/or biological agents on a surface harmless. After the first initial exposure to an atmosphere comprising peroxide vapour, causing the peroxide vapour to condense onto the surface, preferably gaseous base is added such that the gaseous base reacts with the condensed peroxide vapour on the surface (step (II)). After this step, and preferably without removing any of the base present, step (I) and subsequently step (II) is repeated.

Thus in this embodiment, the method of rendering harmless chemical/biological warfare agents on a surface, comprises:
(I) exposing the agents to an atmosphere comprising peroxide vapour, and causing the peroxide vapour to condense on the surface having the chemical/biological agents thereon, preferably in the absence of a gaseous base;
(II) after step (I) adding a gaseous base such that the gaseous base reacts with the condensed peroxide vapour on the surface;
(Ia) after step (II) exposing the agents to an atmosphere comprising peroxide vapour, and causing the peroxide vapour to condense on the surface having the chemical/biological agents thereon;
(Ib) after step (Ia) adding a gaseous base such that the gaseous base reacts with the condensed peroxide vapour on the surface;
(III) removing the gaseous base;
(IV) optionally repeating steps (Ia) and (IIa) at least once, preferably before carrying out step (III).

Preferably once decontamination is complete and step (II) is carried out for the last time, an excess amount of gaseous base is added. Preferably when step (II) is carried out for the last time, gaseous base is added such that the molar amount of base added is at least 2 times, at least 5 times, more preferably at least 10 times, more preferably still at least 20, 50 or 100 times the molar amount of peroxide present.

It is advantageous to add an excess amount of gaseous base the last time step (II) is carried out so that substantially no peroxide is left in the enclosure or system. Preferably, less than 5% by volume of the total gaseous volume of the system is gaseous peroxide, and more preferably less than 2%, more preferably still less than 1%, or less than 0.5%). Preferably, substantially no, and preferably no peroxide vapour is left in the enclosure or system after an excess amount of gaseous base the last time step (II) is carried out.

The present inventors have found that if peroxide is left in the enclosure or system once decontamination is complete, not only does the gaseous base need to be removed from the enclosure or system prior to being able to safely access the surface on which the chemical/biological warfare agents have been rendered harmless, but also the peroxide has to be removed. It has been found to be much more difficult to remove the combined peroxide/base mixture than just a base mixture in the absence of (unreacted) peroxide.

Preferably, after an excess of base has been added in the last step (II), the gaseous base is removed from the enclosure or system. Preferably after removal of the gaseous base, less than 10% by volume, less than 5% by volume, less than 2% by volume, more preferably less than 1% by volume, more preferably still no gaseous base is left in the enclosure or system (chamber, if the reaction is carried out in a chamber, or in the immediate vicinity or the surface if, for example, the surface provides its own chamber, for example the surface is a room to be decontaminated) based on the total amount of gaseous base added in step (II). Most preferably, all the gaseous base is removed. The base gas may be at least partially removed by scrubbing.

Steps (I) and (II) may be repeated once, twice, three, four or more times.

Steps (I), (II) and (III) may be repeated once, twice, three, four or more times.

In one embodiment of the present invention, each time step (I) is repeated, the surface (or the enclosure comprising the surface) is exposed to a reduced amount of peroxide compared to the amount of peroxide to which the surface (or the enclosure comprising the surface) is exposed in the immediately preceding step (I) measured in g of peroxide per cm² of surface.

In another embodiment of the present invention, each time step (I) is repeated, the surface (or the enclosure comprising the surface) is exposed to a reduced amount of peroxide compared to the amount of peroxide to which the surface (or the enclosure comprising the surface) is exposed in the immediately preceding step (I) measured in g of peroxide per m³ of volume.

It will be understood that the amount of peroxide added under the given conditions in the repeated steps (I) will be sufficient to cause the peroxide vapour to condense on the surface to be decontaminated. As the amount of peroxide is reduced, or altered, the conditions in the enclosure and/or system and/or chamber may be altered if necessary.

In an additional, or alternative embodiment of the present invention, each time step (II) is repeated, the surface (or the enclosure comprising the surface) is exposed to a reduced amount of gaseous base compared to the amount of gaseous base to which the surface (or the enclosure comprising the surface) is exposed in the immediately preceding step (II) measured in g of gaseous base per cm² of surface (or measured in g of gaseous base per m³ of volume).

In one embodiment each time step (I) is repeated, the surface (or the enclosure comprising the surface) is exposed to a reduced amount of peroxide compared to the amount of peroxide to which the surface (or the enclosure comprising the surface) is exposed in the immediately preceding step (I) measured in g of peroxide per cm² of surface (or measured in g of peroxide per m³ of volume), and each time step (II) is repeated, the surface (or the enclosure comprising the surface) is exposed to a reduced amount of gaseous base compared to the amount of gaseous base to which the surface (or the enclosure comprising the surface) is exposed in the immediately preceding step (II) measured in g of gaseous base per cm² of surface (or measured in g of gaseous base per m³ of volume).

In another embodiment of the present invention, each time step (I) is repeated, the surface (or the enclosure comprising the surface) is exposed to the same amount of peroxide compared to the amount of peroxide to which the surface (or the enclosure comprising the surface) is exposed in the immediately preceding step (I) measured in g of peroxide per cm² of surface (or measured in g of peroxide per m³ of volume). In an additional, or alternative embodiment of the present invention, each time step (II) is repeated, the surface (or the enclosure comprising the surface) is exposed to the same amount of gaseous base compared to the amount of gaseous base to which the surface (or the enclosure comprising the surface) is exposed in the immediately preceding step (II) measured in g of gaseous base per cm² of surface (or measured in g of gaseous base per m³ of volume).

In one embodiment each time step (I) is repeated, the surface (or the enclosure comprising the surface) is exposed to a reduced amount of peroxide compared to the amount of peroxide to which the surface (or the enclosure comprising the surface) is exposed in the immediately preceding step (I) measured in g of peroxide per cm² of surface (or measured in g of peroxide per m³ of volume), and each time step (II) is repeated, the surface (or the enclosure comprising the surface) is exposed to the same amount of gaseous base compared to the amount of gaseous base to which the surface (or the enclosure comprising the surface) is exposed in the immediately preceding step (II) measured in g of gaseous base per cm² of surface (or measured in g of gaseous base per m³ of volume).

By carrying out the process in an iterative way, and each time decreasing the amount of peroxide, and/or gaseous base used the decontamination of the surface may be optimised, whilst making efficient use of the peroxide, and/or gaseous base.

Preferably, in step (I) the surface comprising the chemical/biological warfare agent is exposed to a pulse of peroxide with a volume dependent on the level of equipment loading in the chamber. A lightly loaded chamber typically requires from 15 to 50 cm³ of peroxide per cubic metre of volume to be decontaminated. Typically a medium loaded chamber is exposed to from 50 to 75 cm³ of peroxide per cubic metre, and a heavily loaded chamber is typically exposed to between 75 to 100 cm³ per cubic metre.

Preferably, in step (II) the surface is exposed to a pulse of gaseous base with a volume dependent on the level of equipment loading in the chamber. Typically a lightly loaded chamber requires from 0.1 to 20 litres of gaseous base (preferably ammonia) per cubic metre of volume to be decontaminated, a medium loaded chamber is typically exposed to from 20 to 40 litres of gaseous base per cubic metre of volume and a heavily loaded chamber is exposed to between 40 to 60 litres of gaseous base per cubic metre of volume.

When the base is added in step (II) it reacts with the condensed peroxide vapour on the surface to be decontaminated, thus it will be understood that the peroxide vapour present in the enclosure and/or system is not purged from the enclosure and/or system before the gaseous base is added in step (II).

Preferably, the surface is exposed to an atmosphere comprising peroxide vapour for less than 1 hour, or less than 40 minutes. Typically the surface is exposed to an atmosphere comprising peroxide vapour for approximately 8 to 60 minutes, from 10 to 25, 30 or 35 minutes, more preferably about 10 minutes. It may be desirable to expose the surface to an atmosphere comprising peroxide vapour for short dwell times in order for the cycle time for the process to be fast.

Preferably, the gaseous base is removed less than 40 minutes, less than 30 minutes, less than 20 minutes, less than 15 minutes after being added. Typically the gaseous base is removed about 10 minutes after being added. It may be desirable to remove the base quickly so that the cycle time for the process is fast.

Preferably no solvents, other than water, are used in the present invention. It will be understood that trace amounts of solvents other than water may be present. Trace amounts of solvents are defined herein as solvents which are present in less than 5% by volume, preferably less than 2% by volume, more preferably less than 0.5% by volume based on the total volume of solvent (including water) present.

Preferably, no co-solvent will be present in the peroxide/water vapour. Preferably the peroxide vapour does not comprise tert-butyl alcohol, acetonitrile, isopropyl alcohol, and mixtures of one or more thereof. Preferably the peroxide vapour does not comprise tert-butyl alcohol, acetonitrile, isopropyl alcohol, tetrahydron, dimethylsulfoxide, acetone, acetaldehyde, propylene oxide, acetamide, diethylamine, dimethoxyethane and mixtures of one or more thereof.

Preferably, the method is carried out under standard atmospheric conditions, for example at a temperature from 10° C. to 35° C., or from 10° C. to 20° C., and at a pressure of approximately 101.325 kPa.

At the end of the process, the enclosure (for example the system or chamber) comprising the surface may be aerated and the decontamination is complete.

In one embodiment of the present invention the surface to be treated is placed in a chamber before carrying out the decontamination process. For example, this may occur when the surface to be treated is a garment or piece of clothing, breathing apparatus, weaponry, medical instruments or any other surface, or apparatus comprising a surface suitable for placing in a chamber. In an alternative embodiment, the surface to be treated may be, for example, the interior of a warehouse, tent, room, aircraft, tank, other vehicle, or the like, whose surfaces (preferably interior surfaces) or items are to be treated.

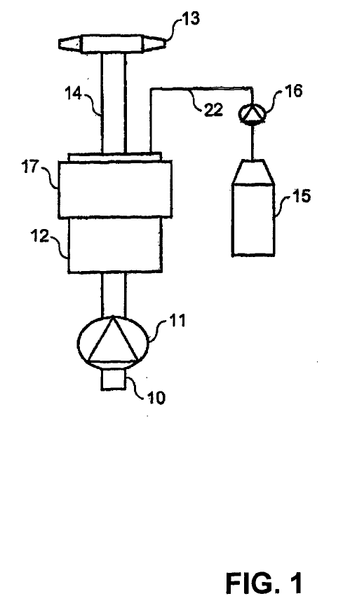

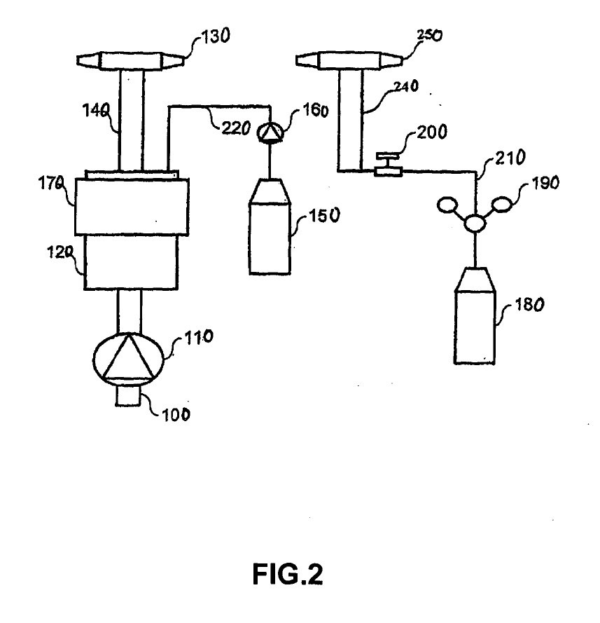

Suitable apparatus for carrying out the present invention is similar to that described in WO 2008/145987. Apparatus suitable for use in the present invention is described with reference to the non-limiting embodiments shown in FIGS. 1 and 2.

FIG. 1 is a diagrammatic illustration of an apparatus for carrying out the present invention.

FIG. 1 shows a schematic of a hydrogen peroxide and water vapour generator suitable for use inside a chamber. Air is drawn into the system through inlet (10) by a fan (11) and passes through an air heater (12) and then an evaporator (17) and eventually leaves the evaporator from nozzles (13) connected to the generator by pipe (14).

Aqueous hydrogen peroxide solution is stored in bottle (15) which is connected by a conduit (22) containing a metering pump (16) to the evaporator to control the flow of the hydrogen peroxide solution to the evaporator (17) where it is flash evaporated into the air flow through the evaporator.

The flash evaporated hydrogen peroxide and water vapour leaves the evaporator through outlet pipe (14). The mixture of hydrogen peroxide vapour, water vapour and air leave the generator through the nozzles (13). In one embodiment the hydrogen peroxide vapour stream is mixed with a high volumetric flow of air from the enclosure before re-entering the enclosure.

The whole process is controlled from a central controller which monitors and adjusts the air flow and rate of evaporation of the aqueous solution of hydrogen peroxide solution. Sensors are provided to measure the hydrogen peroxide vapour concentration so that the metering pump (16) a can operate at the correct time.

FIG. 2 shows a schematic of a hydrogen peroxide and water vapour generator suitable for use inside a chamber, and a separate gaseous ammonia injection stream. Air is drawn into the system through inlet (100) by a fan (110) and passes through an air heater (120) and then an evaporator (170) and eventually leaves the evaporator from nozzles (130) connected to the generator by pipe (140).

Aqueous hydrogen peroxide solution is stored in bottle (150) which is connected by a conduit (220) containing a metering pump (160) to the evaporator to control the flow of the hydrogen peroxide solution to the evaporator (170) where it is flash evaporated into the air flow through the evaporator.

The flash evaporated hydrogen peroxide and water vapour leaves the evaporator through outlet pipe (140). The mixture of hydrogen peroxide vapour, water vapour and air leave the generator through the nozzles (130). In one embodiment the hydrogen peroxide vapour stream is mixed with a high volumetric flow of air from the enclosure before re-entering the enclosure.

Ammonia gas is stored under pressure in a cylinder (180) and released through a pressure control valve (190) and a flow control valve (200) in conduit (210) and then to outlet pipe (240) where the ammonia gas leaves the generator through the nozzles (250).

The whole process is controlled from a central controller which monitors and adjusts the air flow, rate of evaporation of the aqueous solution of hydrogen peroxide solution and the addition of the ammonia gas. Sensors are provided to measure the hydrogen peroxide vapour and ammonia gas concentration so that the metering pump (160) and the valve (200) can operate at the correct time.

It will be understood that nozzles (130) and (250) may be the same nozzle.

The present invention will now be described in relation to the following non-limiting clauses:

1. A method of rendering harmless chemical/biological warfare agents on a surface, comprising:
(I) exposing the agents to an atmosphere comprising peroxide vapour, and causing the peroxide vapour to condense on the surface having the chemical/biological agents thereon;
(II) after step (I) adding a gaseous base such that the gaseous base reacts with the condensed peroxide vapour on the surface;
(III) removing the gaseous base;
(IV) repeating steps (I) and (II) at least once.

2. The method according to clause 1, wherein the gaseous base has the formula NR¹R²R³, wherein R¹, R², R³ are independently selected from the group consisting of a C₁ to C₄ alkyl and hydrogen.

3. The method according to clause 2, wherein the gaseous base which has the formula NR¹R²R³ is ammonia.

4. The method according to any of the preceding clauses, wherein the peroxide is hydrogen peroxide.

b 5. The method according to any of the preceding clauses, wherein step (IV) comprises repeating steps (I), (II) and (III) at least once.

6. The method according to clause 5, wherein step (IV) comprises repeating steps (I), (II), and optionally (III), at least twice.

7. The method according to any of the preceding clauses wherein each time step (I) is repeated, the surface is exposed to a reduced amount of peroxide, measured in g of peroxide per m³ of volume, compared to the amount of peroxide to which the surface is exposed in the immediately preceding step (I).

8. The method according to any of the preceding clauses, wherein the concentration of the peroxide vapour in each step (I) is increased until the dew point of the vapour is exceeded and condensation of the vapour on surfaces takes place.

9. The method according to any of the preceding clauses, wherein the gaseous base is removed in step (III) by scrubbing.

10. The method according to any of the preceding clauses, wherein substantially all of the gaseous base is removed in step (III).

11. The method according to any of the preceding clauses, wherein the chemical/biological warfare agents are one or more of a G-type agent, a V-type, a H-type agent, pathogens, biotoxins, spores and prions.

12. The method according to any of the preceding clauses wherein step (IV) comprises iteratively repeating steps (I), (II), and optionally (III), until the chemical/biological warfare agents originally present on the surface are rendered harmless.

13. The method according to any of the preceding clauses wherein the peroxide vapour comprises water.

14. The method according to clause 13 wherein the peroxide vapour does not comprise a cosolvent.

15. The method according to any of the preceding clauses wherein gaseous base in step (II) is added such that the pH of the condensed peroxide vapour on the surface is in the range of from 9 to 14.

The present invention will now be described with reference to the following non-limited Examples.

### EXAMPLE 1

A 0.3 m³ volume chamber is provided comprising the surface to be decontaminated at 20° C. and 50% RH.

(i) 25 ml of vaporised 30% Hydrogen Peroxide was added to the chamber and left for 30 minutes. These conditions produced a micro-condensate on the surface.

(ii) A further, additional, 20 ml of vaporised 30% Hydrogen Peroxide was added to the chamber and left for 30 minutes. These conditions produced a micro-condensate on the surface

(iii) Step (ii) was repeated until decontamination was complete.

(iv) The sealed chamber was then left for 10 minutes before aerating.

### EXAMPLE 2

A 0.3 m³ volume chamber is provided comprising the surface to be decontaminated at 20° C. and 50% RH.

(I) 15 ml of vaporised 30% Hydrogen Peroxide was added to the chamber and left for 30 minutes. These conditions produced a micro-condensate on the surface.

(II) 1600 ml of NH₃ gas was then added to the chamber over a 1 minute period.

(III) The sealed chamber was then left for 10 minutes before aerating so that substantially all of the gaseous NH₃ was removed.

(IV) Steps (I), (II) and (III) are then repeated, in numerical order once more, until the decontamination was complete.

1. A method of rendering harmless chemical and/or biological warfare agents on a surface, comprising:
(i) exposing the agents to an atmosphere comprising peroxide vapour, and causing the peroxide vapour to condense on the surface having the chemical and/or biological agents thereon;
(ii) after step (i) exposing the agents to an atmosphere comprising further peroxide vapour, and causing the further peroxide vapour to condense on the surface having the chemical and/or biological agents thereon; and
(iii) iteratively repeating step (ii) until the chemical/biological warfare agents originally present on the surface are rendered harmless.

2. The method according to claim 1, wherein the peroxide is hydrogen peroxide.

3. The method according to claim 1 or claim 2, wherein step (iii) comprises performing step (ii) at least a further two times.

4. The method according to claim 1, wherein the surface having the chemical and/or biological agents thereon is in and/or at least part of an enclosure.

5. The method according to claim 4 wherein the surface having the chemical and/or biological agents thereon is placed in the enclosure prior to carrying out step (i).

6. The method according to claim 4, wherein the enclosure is a container, chamber or room.

7. The method according to claim 4, wherein each time step (ii) is repeated, the enclosure comprising the surface is exposed to a reduced amount of peroxide, measured in g of peroxide per m³ of volume, compared to the amount of peroxide to which the enclosure comprising the surface is exposed to in the immediately preceding step (ii).

8. The method according to claim 1, wherein the concentration of the peroxide vapour in each of steps (i) and (ii) is increased until the dew point of the vapour is exceeded and condensation of the vapour on surfaces takes place.

9. The method according to claim 1, wherein the chemical/biological warfare agents are one or more of a G-type agent, a V-type, a H-type agent, pathogens, biotoxins, spores and prions.

10. The method according to claim 1, wherein the peroxide vapour does not comprise a cosolvent.

11. The method according to claim 1, wherein the time period between carrying out step (i) and (ii) is from 5 to 60 minutes.

12. The method according to claim 1, wherein in step (iii) the time period between repeating steps (ii) is from 5 to 60 minutes.

13. The method according to claim 1, wherein the method does not comprise adding base and/or gaseous base and/or an alkaline compound.

14. The method according to claim 1, further comprising adding gaseous base.

15. The method according to claim 14 wherein the gaseous base is added after step (i) such that the gaseous base reacts with the condensed peroxide vapour on the surface.

16. The method according to claim 14, wherein the method further comprises removing at least some, and preferably all, of the gaseous base.

17. The method according to claim 14, comprising:
(I) exposing the agents to an atmosphere comprising peroxide vapour, and causing the peroxide vapour to condense on the surface having the chemical/biological agents thereon;
(II) after step (I) adding a gaseous base such that the gaseous base reacts with the condensed peroxide vapour on the surface;
(III) removing the gaseous base;
(IV) repeating steps (I) and (II) at least once.

18. The method according to claim 14, wherein the gaseous base has the formula NR¹R²R³, wherein R¹, R², R³ are independently selected from the group consisting of a C₁ to C₄ alkyl and hydrogen.

19. The method according to claim 18, wherein the gaseous base which has the formula NR¹R²R³ is ammonia.

20. The method according to claim 17, wherein step (IV) comprises repeating steps (I) and (II) at least once.

21. The method according to claim 20, wherein step (IV) comprises repeating steps (I) and (II) at least twice.

22. The method according to claim 14, wherein each time step (I) is repeated, the surface is exposed to a reduced amount of peroxide, measured in g of peroxide per m³ of volume, compared to the amount of peroxide to which the surface is exposed in the immediately preceding step (I).

23. The method according to claim 16, wherein the gaseous base is removed in step (III) by scrubbing.

24. The method according to claim 17, wherein step (IV) comprises iteratively repeating steps (I) and (II) until the chemical/biological warfare agents originally present on the surface are rendered harmless.

25. The method according to claim 14, wherein gaseous base (preferably in step (II)) is added such that the pH of the condensed peroxide vapour on the surface is in the range of from 9 to 14.
